(19) United States
(12) Patent Application Publication
(10) Pub. No.: US 2016/0287381 A1
(43) Pub. Date: Oct. 6, 2016

(54) LENS INSERTER ASSEMBLY

(71) Applicant: PRESBIBIO, LLC, Irvine, CA (US)

(72) Inventor: Vladimir Feingold, Laguna Niguel, CA (US)

(73) Assignee: PRESBIBIO, LLC, Irvine, CA (US)

(21) Appl. No.: 15/038,700

(22) PCT Filed: Nov. 20, 2014
(86) PCT No.: PCT/US14/66540

§ 371 Date: May 23, 2016

Related U.S. Application Data
(60) Provisional application No. 61/909,519, filed on Nov. 27, 2013.

(51) Int. Cl. A61F 2/16; A61F 2/14; A61F 2/00

(52) U.S. Cl. CPC A61F 2/167; A61F 2/1678; A61F 2/0095; A61F 2/145

(57) ABSTRACT

An apparatus for inserting a lens into an eye includes a handle portion having a proximal end and a distal end, an inserter head assembly slidably coupled to the distal end of the handle portion, a plunger sleeve securely coupled to the handle portion, an actuation sleeve securely coupled to the inserter head assembly, and a plunger coupled to the plunger sleeve and slidably received through the inserter head assembly, wherein the plunger includes a mounting post at a distal end for mounting the lens and a proximal or a distal movement of the actuation sleeve axially extends or retracts the inserter head assembly relative to the plunger. In accordance with other aspects of the present disclosure, an inserter head assembly for a lens insertion apparatus includes a mounting cap and a plunger having a mounting post configured for securing a lens thereon.

### FIELD OF THE INVENTION

The present invention relates generally to ocular surgery. More particularly, the present invention relates to an apparatus and method for inserting a lens into an eye.

### BACKGROUND OF THE INVENTION

The human eye is a specialized sensory organ capable of receiving visual images and transmitting them to the visual center in the brain. Among the main parts of the eye are the cornea, the iris, the lens and the retina. The cornea is the clear window in the front of the eye through which light first passes. The interior surface of the cornea is lined with a single layer of flat, tile-like endothelial cells, whose function is to maintain the transparency of the cornea. The iris is a pigmented muscular curtain located behind the cornea that opens and closes to regulate the amount of light entering the eye through the pupil, an opening at the center of the iris. The lens is a clear structure located behind the cornea that changes shape, or accommodates, to focus light on the back of the eye. The medical term for the lens present in the eye from birth is “crystalline lens.” The retina is a layer of nerve tissue in the back of the eye that senses the light image and transmits it to the brain via the optic nerve.

The eye may be affected by common visual disorders, disease or trauma. A normal, well-functioning eye receives images of objects at varying distances and focuses the images on the retina. Refractive errors occur when the eye cannot properly focus an image on the retina.

Presbyopia is the gradual loss of near vision, which often accompanies the aging process. Presbyopia is an age-related refractive disorder that generally begins to develop when a person reaches the age of 35. The disorder may go unnoticed for several years after its initial onset and can worsen with age. The first symptoms of presbyopia are typically experienced when a person begins to have difficulty reading fine print. Presbyopia is associated with a loss of lens “elasticity,” the ability of the lens to change shape in order to focus on the retina incoming light from objects in near and middle distance ranges. Elasticity is slowly lost as people age, resulting in a slow decrease in the ability of the eye to focus on nearby objects. Presbyopia is a natural part of aging and affects substantially all people at some point in their adult lives. Symptoms of presbyopia can include difficulty reading fine print and blurred vision when transitioning the focus of the eye between near and distant objects.

There are several common treatments for presbyopia. A dedicated pair of reading glasses is one such treatment. Reading glasses provide magnification of near objects to provide for improved vision. However, if a person also needs glasses to focus on distant objects switching between reading glasses and distance glasses can be inconvenient. Another treatment is bifocal glasses, which provide a portion of the glasses lens for assisting with distance vision and a portion for assisting with near vision. While bifocals provide a single pair of glasses for both near and distance vision correction, they can cause disorientation. Contact lenses for the surface of the eye have also been developed which provide vision correction for both near and distance vision. Although these treatments provide vision correction for a person suffering from presbyopia, each requires at least one an additional accessory or pair of contact lenses that must be worn or used daily. Additionally, very small lenses for insertion into the eye are being developed. However, these lenses cannot be handled manually or with conventional tools.

Accordingly, it is desirable to provide an apparatus and method for inserting a lens, particularly a microlens, into the cornea, to improve a patient's presbyopia, for example.

### SUMMARY OF THE INVENTION

The foregoing needs are met, to a great extent, by the present invention, wherein in one aspect an apparatus is provided that in some embodiments includes a design for a lens inserter apparatus and method.

In accordance with one aspect of the present disclosure, an apparatus for inserting a lens into an eye includes a handle portion having a proximal end and a distal end, an inserter head assembly slidably coupled to the distal end of the handle portion, a plunger sleeve securely coupled to the handle portion, an actuation sleeve securely coupled to the inserter head assembly, and a plunger coupled to the plunger sleeve and slidably received through the inserter head assembly, wherein the plunger includes a mounting post at a distal end for mounting the lens and a proximal or a distal movement of the actuation sleeve axially extends or retracts the inserter head assembly relative to the plunger.

In accordance with other aspects of the present disclosure, an inserter head assembly for a lens insertion apparatus includes a mounting cap, a plunger having a mounting post configured for securing a lens thereon, the plunger being slidably received through the mounting cap, a top leaf extending distally from the mounting cap, and a bottom leaf extending distally from the mounting cap, the top leaf and the bottom leaf biased to abut with at least a portion of the plunger sandwiched between.

In accordance with yet other aspects of the present disclosure, a sterile packaging assembly includes an inserter head assembly having a mounting cap, a plunger slidably received through a mounting cap, a top leaf extending distally from the mounting cap, and a bottom leaf extending distally from the mounting cap, the top leaf and the bottom leaf biased to abut with at least a portion of the plunger sandwiched between, a stopper mounted to the inserted head assembly, and a vile for receiving the stopper with mounted head assembly therein.

In accordance with yet other aspects of the present disclosure, a method for insertion of a lens into a corneal pocket includes providing a lens insertion apparatus having a handle portion having a proximal end and a distal end; an inserter head assembly slidably coupled to the distal end of the handle portion; and a plunger slidably received through the inserter head assembly; and mounting a lens onto a mounting post at a distal end of the plunger.

There has thus been outlined, rather broadly, certain embodiments of the invention in order that the detailed description thereof herein may be better understood, and in order that the present contribution to the art may be better appreciated. There are, of course, additional embodiments of the invention that will be described below and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of embodiments in addition to those described and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

FIGS. 1-4 illustrate a front view of a lens inserter apparatus in various stages of use relative to the cornea of an eye, in accordance with certain aspects of the present disclosure;

FIG. 5 illustrates a sectional view of the anterior portion of an eye with an intracorneal lens disposed therein, in accordance with certain aspects of the present disclosure;

FIG. 6 illustrates a perspective view of a lens inserter apparatus, in accordance with certain aspects of the present disclosure;

FIG. 7 illustrates a top perspective view of a plunger for use in a lens inserter apparatus, in accordance with certain aspects of the present disclosure;

FIG. 8 illustrates a bottom perspective view of the plunger shown in FIG. 7, in accordance with certain aspects of the present disclosure;

FIG. 9 illustrates a perspective view of component parts configured for use in a lens inserter apparatus, in accordance with certain aspects of the present disclosure;

FIG. 10 illustrates a longitudinal sectional view of a lens inserter apparatus, in accordance with certain aspects of the present disclosure;

FIG. 11 illustrates a perspective view of component parts configured for use in a lens inserter apparatus, in accordance with certain aspects of the present disclosure;

FIG. 12 illustrates another perspective view of the lens inserter apparatus shown in FIG. 6 in a particular state of use, in accordance with certain aspects of the present disclosure;

FIG. 13 illustrates another longitudinal sectional view of a lens inserter apparatus shown in FIG. 10 in a particular state of use, in accordance with certain aspects of the present disclosure;

FIG. 14 illustrates an enlarged view of an inserter head assembly in a particular state of use, in accordance with certain aspects of the present disclosure;

FIG. 15 illustrates an enlarged view of an inserter head assembly in a particular state of use, in accordance with certain aspects of the present disclosure;

FIG. 16 illustrates an enlarged view of an inserter head assembly in a particular state of use, in accordance with certain aspects of the present disclosure;

FIG. 17 illustrates an enlarged view of an inserter head assembly in a particular state of use, in accordance with certain aspects of the present disclosure;

FIG. 18 illustrates an enlarged view of an inserter head assembly in a particular state of use, in accordance with certain aspects of the present disclosure;

FIG. 19 illustrates a packaging assembly for an inserter head assembly, in accordance with certain aspects of the present disclosure;

FIG. 20 illustrates component parts of a packaging assembly for an inserter head assembly, in accordance with certain aspects of the present disclosure; and

FIG. 21 illustrates component parts of a packaging assembly for an inserter head assembly, in accordance with certain aspects of the present disclosure.

### DETAILED DESCRIPTION

The invention will now be described with reference to the drawing figures, in which like reference numerals refer to like parts throughout.

Various aspects of a lens inserter assembly may be illustrated by describing components that are coupled, attached, and/or joined together. As used herein, the terms “coupled”, “attached”, and/or “joined” are used to indicate either a direct connection between two components or, where appropriate, an indirect connection to one another through intervening or intermediate components. In contrast, when a component is referred to as being “directly coupled”, “directly attached”, and/or “directly joined” to another component, there are no intervening elements present.

Relative terms such as “lower” or “bottom” and “upper” or “top” may be used herein to describe one element's relationship to another element illustrated in the drawings. It will be understood that relative terms are intended to encompass different orientations of a lens inserter assembly in addition to the orientation depicted in the drawings. By way of example, if aspects of a lens inserter assembly shown in the drawings are turned over, elements described as being on the “bottom” side of the other elements would then be oriented on the “top” side of the other elements. The term “bottom” can therefore encompass both an orientation of “bottom” and “top” depending on the particular orientation of the apparatus.

Various aspects of a lens inserter assembly may be illustrated with reference to one or more exemplary embodiments. As used herein, the term “exemplary” means “serving as an example, instance, or illustration,” and should not necessarily be construed as preferred or advantageous over other embodiments of a lens inserter assembly disclosed herein.

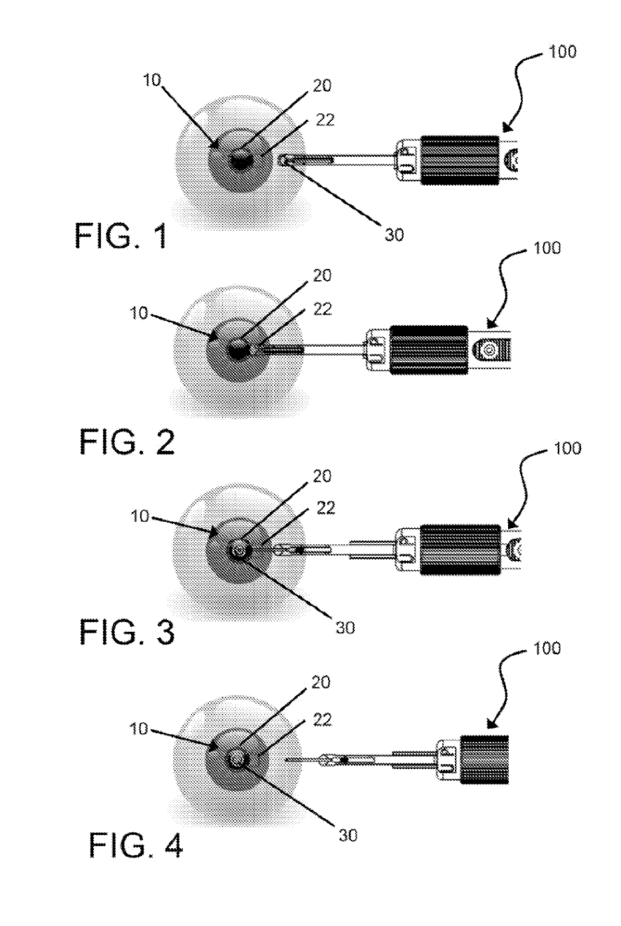

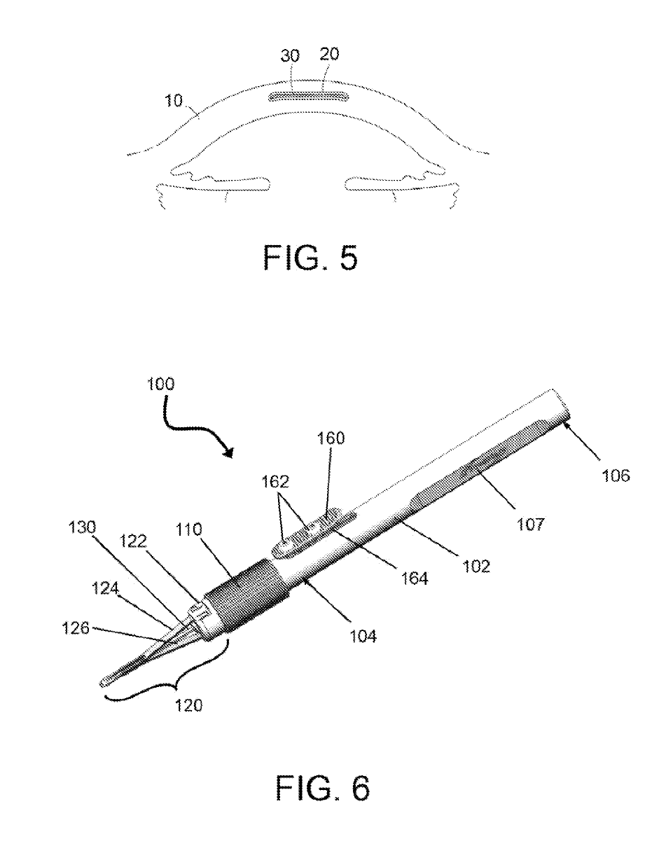

FIGS. 1-4 illustrate aspects of a lens inserter apparatus 100 shown in various positions of use in accordance with the present invention. As shown, a cornea 10 may have a corneal pocket 20 formed by a laser surgery apparatus as described, for example, in U.S. patent application Ser. No. 13/222,042, entitled Method for Laser Cutting a Corneal Pocket, the entirety of which is incorporated herein by reference. The corneal pocket 20 may be formed by photo disruption using a laser beam from a laser source, for example, and formed with a thickness and shape that conforms to the surfaces of an intracorneal lens 30 (see also FIG. 5). The interior surfaces of the corneal pocket 20 may be convex, concave, planar or irregular. The edges of the corneal pocket 20 may form an outline having various shapes depending on the desired outcome and the shape of the intracorneal lens 30. The various configurations of corneal pockets can be adapted to be used with lenses of various shapes and sizes.

The corneal pocket 20 may be configured to facilitate the insertion of the lens 30 and minimize the size of the incision for improved post-surgical healing of the cornea. For example, the corneal pocket 20 may also include an entry channel 22 that may be cut into the cornea 10 after the corneal pocket 20 is formed. Entry channel 22 may facilitate the insertion of the intracorneal lens 30 into the corneal pocket 20.

FIGS. 1-4 illustrate generally the method for location and placement of the intracorneal lens 30 into the corneal pocket 20. The lens 30 may be a microlens that is a disc shaped lens having a refractive zone in the periphery designed to improve near vision problems associated with presbyopeia and a central zone that is plano, or non-refractive, designed to maintain distance vision. The overall diameter of the lens may be 3-5 mm, for example, and the lens may have a central hole as small as 0.5 mm for mounting and/or handling of the lens 30. As shown in FIG. 1, the lens inserter apparatus 100, with the lens 30 secured toward a distal end thereof, may be positioned for an approach toward the entry channel 22. As shown in FIG. 2, the lens inserter apparatus 100 with lens 30 secured thereto may be guided into the entry channel 22. As shown in FIG. 3, the lens inserter apparatus 100 may be actuated for release and placement of the lens 30 into the corneal pocket 20 at or near the center of the cornea 10. As shown in FIG. 4, with the lens 30 placed in position, the lens inserter apparatus 100 may be withdrawn through the entry channel 22.

FIG. 6 illustrates a perspective view of the lens inserter apparatus 100 in accordance with aspects of the present disclosure. The lens inserter apparatus 100 includes a handle portion 102, which may be a cylindrical tube having a distal end portion 104 and a proximal end 106. The handle portion 102 may include haptic features, such as reduced thickness area 107, for example, to facilitate an ergonomic efficiency in the handling of the apparatus 100 during a procedure. A coupling sleeve 110 may be provided at the distal end portion 104 of the handle portion 102 for slidably coupling an inserter head assembly 120 to the handle portion 102.

The inserter head assembly 120 includes a pair of leaves, referred to herein as a top leaf 124 and a bottom leaf 126, which may be coupled to or integrally formed to extend from the mounting cap 122. The thickness of each of leaves 124 and 126 may be in the range of approximately 50 microns to 200 microns for a combined thickness in a range of 100 microns to 400 microns. The leaves 124 and 126 may be formed from stainless steel or any other suitable non corrosive material. The top leaf 124 and the bottom leaf 126 are configured to be biased together, such that an inner surface of the top leaf 124 is in contact with an inner surface of the bottom leaf 126, and are preferably made from a flexible, resilient material. A plunger 130 slidably extends through the mounting cap 122 with a portion of the plunger being sandwiched by the leaves 124 and 126.

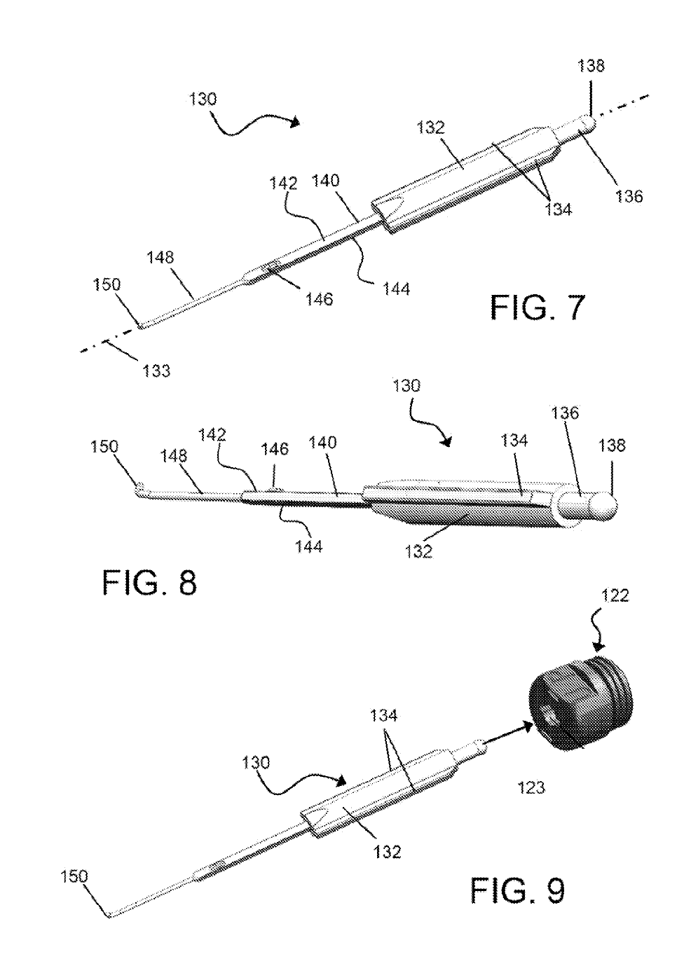

As shown in greater detail in FIGS. 7 and 8, the plunger 130 may be formed with a main body portion 132 having a central axis 133 and lateral wings 134 running longitudinally along both sides of the body portion 132. A mounting member 136 with a ball end 138 may be concentrically situated about the central axis 133 and extend proximally from the main body portion 132. A biasing arm 140 may extend distally from the main body portion 132 along the central axis 133. The biasing arm 140 may have a substantially flat upper biasing surface 142 and a substantially flat lower biasing surface 144 for efficiently sliding between the distal end portions of the top leaf 124 and the bottom leaf 126. A biasing protrusion 146 may be provided toward a distal end of the upper biasing surface 142 of the biasing arm 140. Finally, a mounting arm 148 may extend from the distal end of biasing arm 140. The mounting arm 148 may be substantially cylindrical in shape and configured to extend concentrically along the central axis 133 until terminating in a curved portion that forms a mounting post 150. The mounting post may The mounting post 150 may be tapered or stepped, for example, to assist in the mounting and retention of the lens 30 thereon.

As shown in FIG. 9, the mounting cap 122 may be formed to have a through-hole 123 shaped for slidaby receiving the main body portion 132 of the plunger 130. The through-hole 123 may be shaped to accept the lateral wings 134 of the plunger 130 in a keyed fashion such that rotation of the mounting cap 122 will result in reciprocal rotation of the plunger 130 and hence recipricol rotation of the mounting post 150.

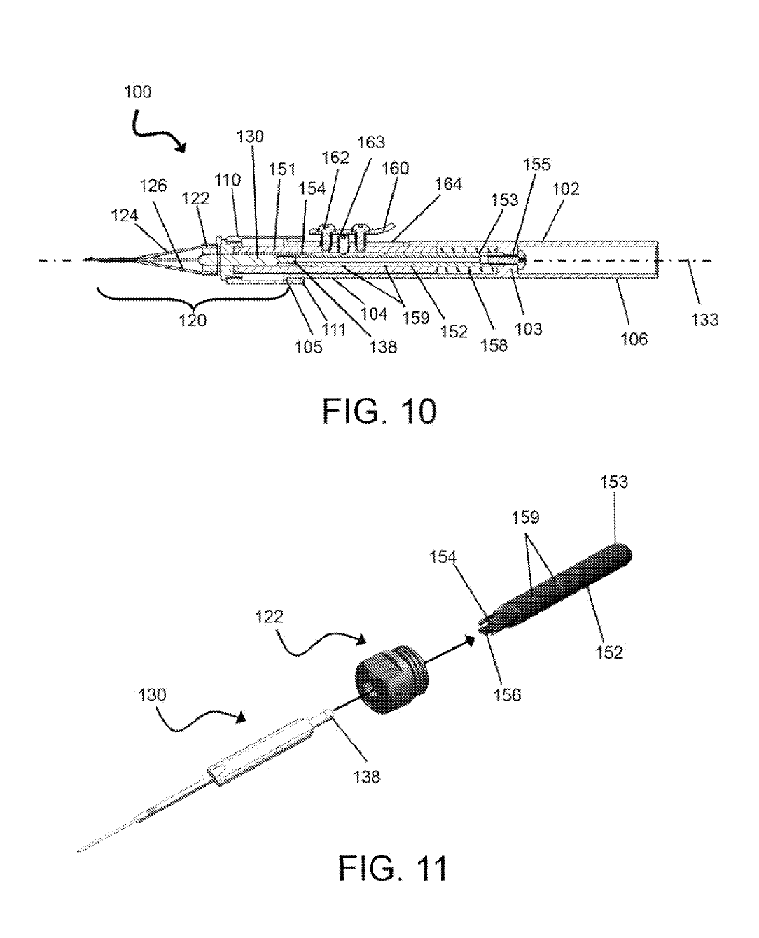

As shown in FIG. 10, the inserter head assembly 120, comprising the mounting cap 122, the leaves 124 and 126, and the plunger 130, may form an integral unit that is separately attachable to the handle portion 102 of the inserter apparatus 100. In accordance with certain aspects of the present disclosure, the inserter head assembly 120 may thus form a disposable unit, if desired, or may be easily removed from the inserter apparatus 100 in order to have the parts cleaned and disinfected.

An actuation sleeve 151 may be slidably housed in the hollow interior distal end portion 104 of the handle portion 102. The actuation sleeve 151 is concentrically arranged about the central axis 133 and configured to couple with the mounting cap 122 of the inserter head assembly 120 so that the plunger 130 is slidably situated inside of the actuation sleeve 151. An actuator 160 may be coupled to the actuation sleeve 151 via a suitable securing means, such as by lock screws 162 or posts. The actuator 160 may be textured to provide friction between the actuator and an operator's finger (e.g., see FIG. 6).

As shown in FIGS. 10 and 11, a plunger sleeve 152 may be provided that is housed in the cylindrical interior of the handle portion 102. The plunger sleeve 152 may be cylindrical in shape having a proximal end 153, a distal end 154, and an outer circumference substantially equal to an inner circumference of the actuation sleeve 151. The proximal end 153 of the plunger sleeve 152 may be internally threaded for securely mounting the plunger sleeve to an internal stop 103 provided inside of handle portion 102. Any suitable securing mechanism, such as a screw 155 may be used to secure the plunger sleeve 152 to the handle portion 102. The distal end 154 of the plunger sleeve 154 is configured to form a socket 156 for receiving and securing the ball end 138 of the plunger 130.

To mount the inserter head assembly 120 to the handle portion 102, the ball end 138 of the plunger 130 is forced to matingly engage the socket 156 of the plunger sleeve 152. The coupling sleeve 110, which is mounted to the distal end portion 104 of the handle portion 102 may then be secured to the mounting cap 122 via a threaded connection, for example. Locking detent 111 may be provided at the proximal end of the coupling sleeve 110 to work in tandem with end stop 105 provided at the distal end portion 104 of the handle portion 102 to prevent disengagement of the inserter head assembly 120 from the handle portion 102.

As assembled, the mated plunger 130 and plunger sleeve 152 are axially aligned along the central axis 133 and secured to the handle portion 102 in a manner to prevent axial movement of the plunger 130 along the central axis 133 relative to the handle portion 102. The actuation sleeve 151, which is secured to the inserter head assembly 120, is thus slidably arranged concentrically around the mated plunger 130 and plunger sleeve 152. A biasing spring 158 may be provided interior to the handle portion 102 to bias the actuation sleeve 151 and thus the coupled inserter head assembly 120 toward the distal end of the inserter apparatus 100. As noted previously, the locking detent 111 at the proximal end of the coupling sleeve 110 works in tandem with end stop 105 provided at the distal end portion 104 of the handle portion 102 prevents disengagement of the inserter head assembly 120 from the handle portion 102 while also defining a travel boundary for axial movement of the actuation sleeve 151 in a distal direction relative to the securely coupled plunger 130 and handle portion 102. The biasing spring 158 and/or an axial length of the coupling sleeve 110, for example, may define a second travel boundary for axial movement of the actuation sleeve 151 in the proximal direction relative to the coupled plunger 130 and handle portion 102.

In other aspects of the present disclosure, as shown in FIGS. 10 and 11, the actuation sleeve 151 may be configured to mount a spring loaded set screw 163 therethrough. The plunger sleeve 152 may be formed with radial locking grooves 159 that are engaged by the set screw 163 to lock the actuation sleeve 151 at predetermined positions of extension or retraction with respect to the plunger sleeve 152.

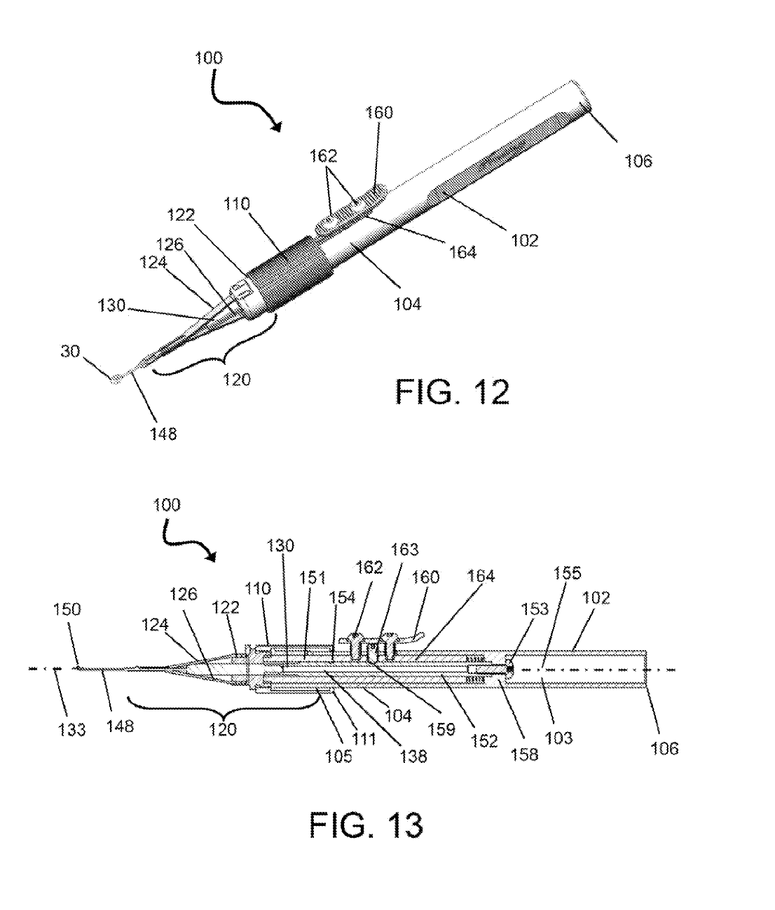

FIGS. 6 and 10 illustrate the apparatus 100 in a certain position of use, namely a storage position in which the plunger 130 is retracted substantially completely in a distal direction with respect to the inserter head assembly 120. In this position, the lens 30 may be effectively secured by virtue of the mounting post 150 and the clamped distal ends of the top leaf 124 and bottom leaf 126. To use the apparatus 100 in an insertion procedure, as shown in FIGS. 12 and 13, the actuator 160 is configured to move the actuation sleeve 151 coupled to the inserter head assembly 120 proximally relative to the coupled plunger 130 and handle portion 102. A channel groove 164 is provided in the wall of the cylindrical handle portion 102 to permit free movement of the actuator 160 relative to the handle portion 102. As the inserter head assembly 120 is retracted proximally relative to the plunger 130, the mounting arm 148 and mounting post 150 of the plunger 130 are not retracted but, rather, are exposed so that the lens 30 may be easily inserted into the corneal pocket 20, as discussed above and shown in FIGS. 1-4. The actuation sleeve 151 may thus be retracted against the force of the biasing spring 158 until the set screw 163 engages the proximal locking groove 159 to lock the assembly in a position for release of the lens 30.

The lens 30 is thus effectively secured in the inserter apparatus 100 until properly positioned through the entry channel 22 and into the corneal pocket 20. Once in position, the inserter head assembly 120 may be retracted using the actuator 160 while leaving the lens 30 mounted on the mounting post 150 in an unsecured state ready for release. The lens 30 may be placed into the exact predetermined position in the corneal pocket 20. With the lens in position, a slight pressure applied posteriorly to the apparatus 100 will disengage the mounting post 150 from the lens 30, dismounting the lens in the predetermined position. The apparatus 100 may be rotated slightly so that the mounting post 150 aligns with a transverse dimension of the corneal entry channel for easy removal and complete withdrawal of the apparatus 100 through the entry channel 22.

FIGS. 14-18 are enlarged illustrations of the inserter head assembly 120 to more particularly describe aspects of the lens inserter apparatus 100 during an actuation procedure. As shown in FIG. 14, in the secured or storage position, the lens 30 may be mounted on the mounting post 150 such that the mounting post 150 extends through a center aperture provided in the lens 30. The inserter head assembly 120 remains in a fully extended position with the plunger effectively sandwiched between the leaves 124 and 126. The biasing spring 158 biases the inserter head assembly 120 distally until the inserter head assembly 120 is retracted against the force of the spring via actuation of the actuator 160.

The bottom leaf 126 has a generally flat elongate upper surface 170, which is configured to have a substantially circular, lens shaped portion 172 at the free end. A bottom leaf channel 174 may be formed in the bottom leaf 126 for accommodating the mounting arm 148 of the plunger 130. Similarly, the top leaf 124 has a generally flat elongate lower surface 176 configured with a paddle portion 178 at the free end. A top leaf channel 180 may be formed in the top leaf 124. As shown in FIG. 14, in the secured position the lens 30 is secured on the mounting post 150 with the mounting arm 148 accommodated sufficiently in the bottom leaf channel 174 so that the lens 30 may rest flush on the circular, lens shaped portion 172 of the bottom leaf 126. The biasing protrusion 146 on the biasing arm 140 of the plunger 130 may be configured to rest in the space between the cantilevered top leaf 124 and bottom leaf 126 just proximal to where the elongate lower surface 176 of the top leaf 124 and the elongate upper surface 170 of the bottom leaf 126 are forced together in the secured state. With the top leaf 124 and the bottom leaf 126 biased in this manner to force the elongate upper surface 170 of the bottom leaf 126 to contact the elongate lower surface 176, at least a portion of the lens 30 may be secured between the paddle portion 178 of the top leaf 124 and the lens shaped portion 172 of the bottom leaf 126. With the lens 30 thus secured and protected, as shown in FIGS. 1-4, the inserter apparatus 100 may be inserted through the entry channel 22 to locate the secured lens 30 in the desired location in the corneal pocket 20.

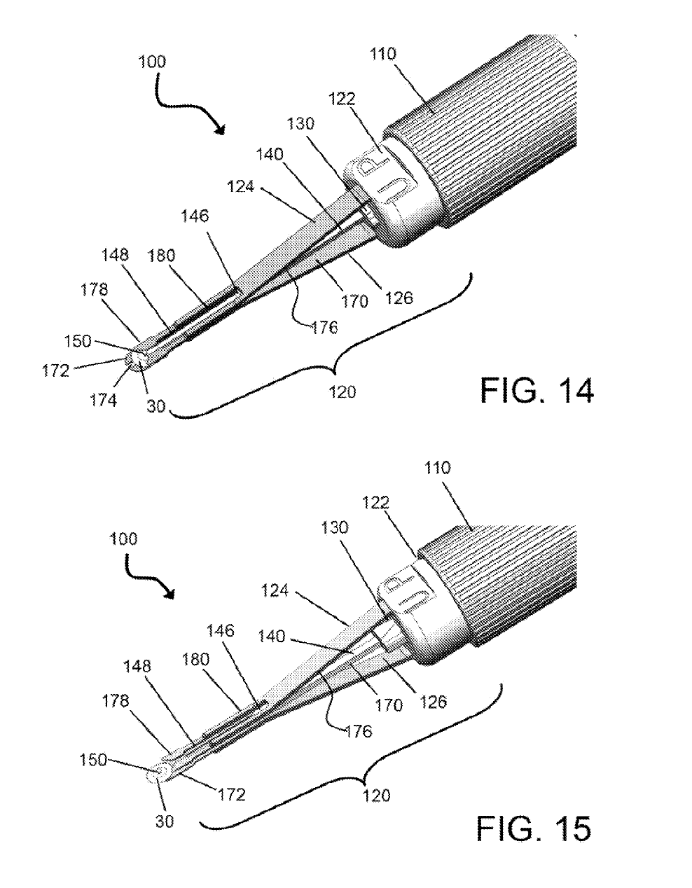

FIG. 15 illustrates an enlarged view of the inserter head assembly 120 just after the actuator 160 is actuated to begin retraction of the inserter head assembly 120 proximally. Because the plunger 130 is configured to remain in position as the inserter head assembly 120 is retracted, the top leaf 124 and the bottom leaf 126 are forced to separate by passing over the biasing protrusion 146 of the biasing arm 140. The biasing protrusion 146 may be configured with sufficient longitudinal length to ensure that the top leaf 124 and the bottom leaf 126 remain separated long enough for the lens 30 to be clear of at least the paddle portion 178 of the top leaf 124 before the top leaf 124 and bottom leaf 126 are biased into a closed position again. In addition, the leading and trailing edges of the biasing protrusion 146 may be tapered or angled to allow a smooth transition from an open to a closed position and vice versa.

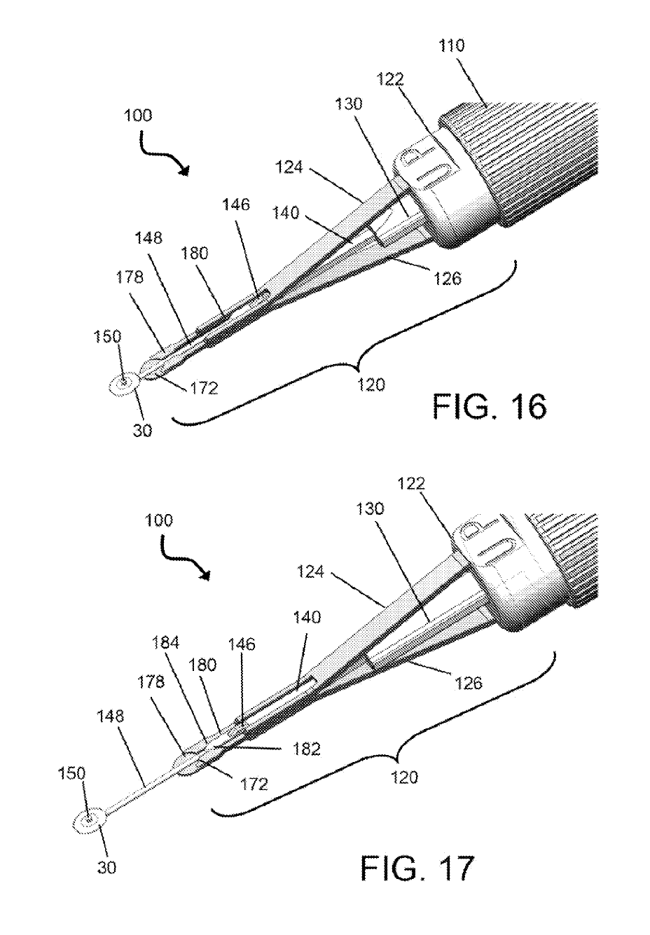

As shown in FIG. 16, as the inserter head assembly 120 continues to retract relative to the plunger 130, the biasing protrusion 146 along with the mounting arm 148 is eventually accommodated in the open space of the top leaf channel 180 and the bottom leaf channel 174. Accordingly, with the biasing protrusion 146 no longer in abutment with the top leaf 124, the leaves 124 and 126 are biased back into a closed, clamped position. The biasing arm 140 and the mounting arm 148 are configured to slide through the open space formed by the top leaf channel 180 and the bottom leaf channel 174 as the inserter head assembly 120 is retracted.

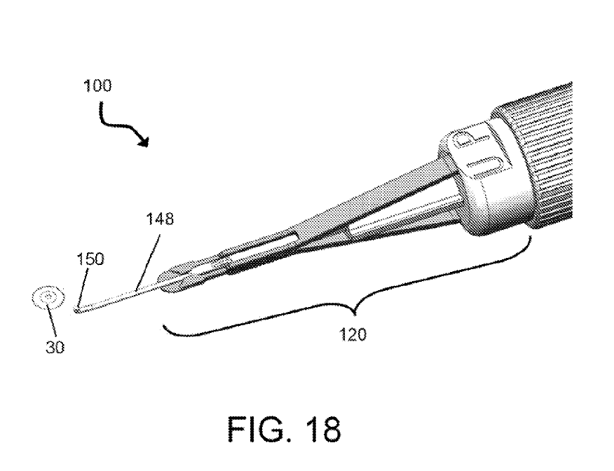

As shown in FIG. 17, the inserter head assembly 120 may continue retracting to further expose the mounting arm 148 and the lens 30 mounted on the mounting post 150 at the end of the mounting arm 148. Detents 182 may be formed in one or both of the top leaf channel 180 and the bottom leaf channel 174 to define an end stop abutment for limiting the retraction motion. In this manner, a tapered transition area 184 where the biasing arm 140 yields in dimension to the mounting arm 148 may be configured to abut the detent 182. As noted previously, the set screw 163 may engage the locking groove 159 in this fully retracted position to allow the practitioner to lessen or release entirely the force being applied to the actuator 160. As described with respect to FIGS. 1-4, with the inserter head assembly 120 fully retracted from the insertion position, the lens 30 may easily be dismounted from the mounting post 150 into the corneal pocket 20 and the inserter apparatus 100 fully withdrawn so that the mounting arm 148 is withdrawn from the entry channel 22 (see also FIG. 18).

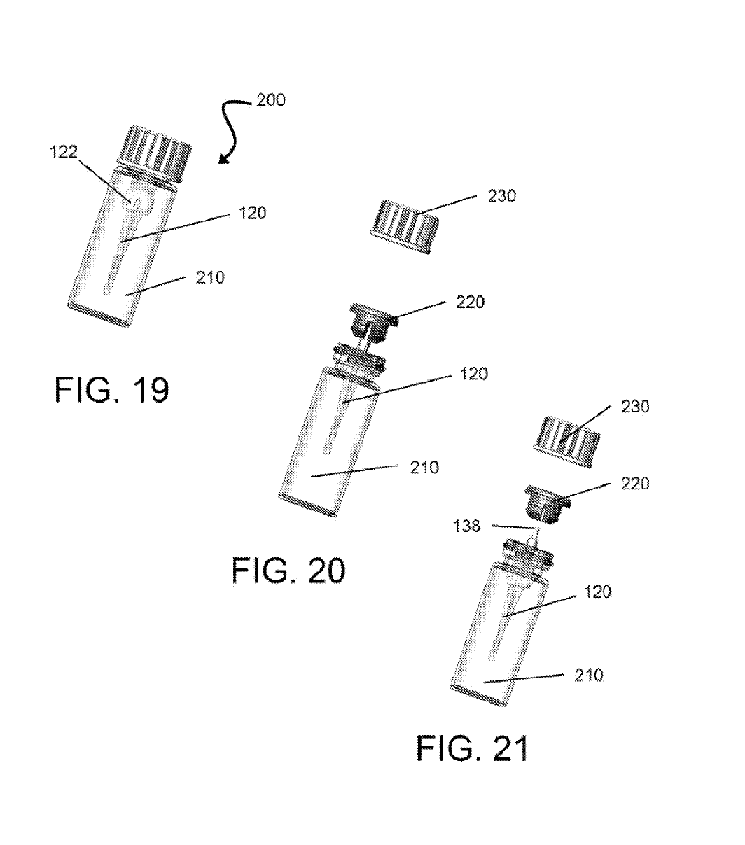

FIGS. 19-21 illustrate a packaging assembly 200 for the inserter head assembly 120. As shown, a suitable containment device, such as a vile 210, may be configured to hold the inserter head assembly 120. For example, a stopper 220 may be mounted onto the ball end 138 of the plunger such that the inserter head assembly may be securely mounted into an interior portion of the vile 210. A cap 230 may be used to secure the stopper 220 and inserter head assembly 120 from dislodgement during transport, maintenance and/or use. The packaging assembly 200 may be sterilized, initially without the lens 30. The sterilized lens 30 may then be mounted into the inserter head assembly 120 and secured, the inserter head assembly 120 with the secured lens 30 being placed back into the vile 210 to form a sterilized, packaging assembly 200 for use with the inserter apparatus 100. To use a sterilized inserter head assembly 120, the cap 230 and stopper 220 may be removed to expose the ball end 138 of the plunger 130. A separate sterilized handle portion 102 may be mounted onto ball end 138 by mating the socket 156 of plunger sleeve 152 with the ball end 138 of the plunger 130 and tightening the coupling sleeve 110 to the mounting cap 122 of the inserter head assembly 120.

The many features and advantages of the invention are apparent from the detailed specification, and thus, it is intended by the appended claims to cover all such features and advantages of the invention, which fall within the true spirit, and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

What is claimed is:

1. An apparatus for inserting a lens into an eye comprising:
a handle portion having a proximal end and a distal end;
an inserter head assembly slidably coupled to the distal end of the handle portion;
a plunger sleeve securely coupled to the handle portion;
an actuation sleeve securely coupled to the inserter head assembly; and
a plunger coupled to the plunger sleeve and slidably received through the inserter head assembly, wherein the plunger includes a mounting post at a distal end for mounting the lens and a proximal or a distal movement of the actuation sleeve axially extends or retracts the inserter head assembly relative to the plunger.

2. The apparatus of claim 1, wherein the inserter head assembly further comprises a top leaf and a bottom leaf extending distally from a mounting cap, the top leaf and the bottom leaf biased to abut.

3. The apparatus of claim 2, wherein the plunger includes a main body portion having a central axis and a pair of lateral wings extending longitudinally along opposite sides of the main body portion.

4. The apparatus of claim 3, wherein the plunger further includes a mounting member having a ball end extending proximally from the main body portion and a biasing arm extending distally from the main body portion.

5. The apparatus of claim 4, wherein the biasing arm includes a substantially flat upper biasing surface and a substantially flat lower biasing surface for sliding between distal end portions of the top leaf and the bottom leaf.

6. The apparatus of claim 5, wherein the plunger further includes a biasing protrusion toward a distal end of the upper biasing surface, the biasing protrusion configured to separate the top leaf and the bottom leaf when abutting the top leaf during retraction of the inserter head assembly.

7. The apparatus of claim 6, wherein the top leaf has a top leaf channel for receiving the biasing protrusion following a predetermined distance of retraction of the inserter head assembly relative to the plunger.

8. The apparatus of claim 4, wherein the plunger sleeve includes a socket portion for mating with the ball end of the mounting member.

9. The apparatus of claim 1, wherein an actuator is coupled to the actuation sleeve.

10. The apparatus of claim 9, wherein the handle includes a groove channel configured to accommodate actuation of the actuator.

11. The apparatus of claim 2, wherein the bottom leaf further includes a lens shaped portion at a distal end and the top leaf further includes a paddle portion at a distal end, and wherein when in a secured position, the lens is mounted on the mounting post and sandwiched between the lens shaped portion and the paddle portion.

12. An inserter head assembly for a lens insertion apparatus, the inserter head assembly comprising:
a mounting cap;
a plunger having a mounting post configured for securing a lens thereon, the plunger being slidably received through the mounting cap;
a top leaf extending distally from the mounting cap; and
a bottom leaf extending distally from the mounting cap, the top leaf and the bottom leaf biased to abut with at least a portion of the plunger sandwiched between.

13. The apparatus of claim 12, wherein the plunger includes a main body portion having a central axis and a pair of lateral wings extending longitudinally along opposite sides of the main body portion; and wherein the mounting cap has a through- hole configured in the shape of the main body portion.

14. The apparatus of claim 13, wherein the plunger further includes a mounting member having a ball end extending proximally from the main body portion and a biasing arm extending distally from the main body portion.

15. The apparatus of claim 14, wherein the biasing arm includes a substantially flat upper biasing surface and a substantially flat lower biasing surface for sliding between distal end portions of the top leaf and the bottom leaf.

16. The apparatus of claim 15, wherein the plunger further includes a biasing protrusion toward a distal end of the upper biasing surface, the biasing protrusion configured to separate the top leaf and the bottom leaf when abutting the top leaf.

17. The apparatus of claim 16, wherein the top leaf has a top leaf channel for receiving the biasing protrusion following a predetermined distance of retraction of the mounting cap relative to the plunger.

18. A sterile packaging assembly, comprising:
an inserter head assembly having a mounting cap, a plunger slidably received through a mounting cap, a top leaf extending distally from the mounting cap, and a bottom leaf extending distally from the mounting cap, the top leaf and the bottom leaf biased to abut with at least a portion of the plunger sandwiched between;
a stopper mounted to the inserted head assembly; and
a vile for receiving the stopper with mounted head assembly therein.

19. The sterile packaging assembly of claim 18, further comprising:
a cap for securing the stopper and mounted head assembly in the vile.

20. A method for insertion of a lens into a corneal pocket, the method comprising:
providing a lens insertion apparatus that includes:
a handle portion having a proximal end and a distal end;
an inserter head assembly slidably coupled to the distal end of the handle portion;
and a plunger slidably received through the inserter head assembly;
mounting a lens onto a mounting post at a distal end of the plunger.
